(19) United States
(12) Patent Application Publication
(10) Pub. No.: US 2021/0340226 A1
(43) Pub. Date: Nov. 4, 2021

(54) HUMAN MEDICAL PROPHYLAXIS OF CORONAVIRIDAE PATHOGENIC INFECTION BY TOPICAL APPLICATION OF IMMUNE CORONAVIRIDAE IMMUNOGLOBULIN A

(71) Applicants: Robert Scott Olson, Ann Arbor, MI (US); Michael R. Simon, Ann Arbor, MI (US)

(72) Inventors: Robert Scott Olson, Ann Arbor, MI (US); Michael R. Simon, Ann Arbor, MI (US)

(21) Appl. No.: 17/236,213

(22) Filed: Apr. 21, 2021

Related U.S. Application Data
(60) Provisional application No. 63/018,534, filed on May 1, 2020.

(51) Int. Cl. C07K 16/10; A61P 31/14

(52) U.S. Cl. CPC C07K 16/10; A61P 31/14

(57) ABSTRACT

A method of inhibiting or treating infection by Coronaviridae virus of a susceptible host is provided that includes the administration of immune anti-Coronaviridae secretory IgA having a recombinant secretory component to at least one of tissue of the susceptible host. The administration being prior to, or after the susceptible host is exposed to the Coronaviridae virus. A composition for such administration is also provided.

### RELATED APPLICATIONS

This application claims priority benefit of U.S. Provisional Application Ser. No. 63/018,534 filed May 1, 2020; the contents of which are hereby incorporated by reference.

### FIELD OF THE INVENTION

The present invention relates to human medical prophylaxis of Coronaviridae virus infection of immunoglobulin (lg) A, and in particular to Coronaviridae viral infections of the lower respiratory tract, nose, throat and eye by administering doses of IgA rich in anti-Coronaviridae immunoglobulin A, the indications including SARS-CoV-2 infection.

### BACKGROUND OF THE INVENTION

The Coronaviridae family of viruses infect many species of animals, including humans. The prototype murine coronavirus strain JHM was reported in 1940s. Some of the animal Coronaviridae viruses that are pathogenic include porcine transmissible gastroenteritis virus (TGEV), bovine coronavirus (BCoV), and avian infectious bronchitis viruses (IBV). In 2003 Coronaviridae viruses gained attention when it became clear that a newly discovered human coronavirus was responsible for severe acute respiratory syndrome (SARS). With the occurrence of the SARS epidemic, coronaviruses became an emerging pathogen.

A SARS coronavirus (SARS-CoV-2) emerged in 2019, was designated COVID-19 and reached pandemic proportions. The ease with which this infection spreads is surprising. The SARS-CoV and SAR-CoV-2 viruses attach to cells targeted for infection via its surface spike (S) protein which may play a role in pathogenesis by inducing interleukin-8 (IL-8) in the lungs via activations of MAPK and AP (Y. J. Chang et al. J. Immunol. 2004,173: 7602-7614). The spike protein binds to the cells' angiotensin II Type 2 receptors. W. Li et al. Nature 2003, 426:450-454.

Immunoglobulins (also called antibodies) are a group of structurally related proteins composed of heavy and light chains. These proteins are categorized as IgM, IgG, IgD, IgE, and IgA depending upon the characteristics of the constant regions of their heavy chains (designated μ, y, Ii, E, and a, respectively). The variable regions of the heavy chains along with the variable regions of the light chains determine the molecular (antibody) specificity of the complete molecule. These molecules are secreted by B lymphocytes in response to signals from other components of the immune system. Their function is to prevent and combat infection by viruses and bacteria.

Purified IgG from pooled human plasma is administered intravenously in humans to treat a variety of conditions. In the purification, a fraction rich in IgA is considered an unwanted by-product, since intravenous administration of IgA containing immunoglobulin can cause life threatening anaphylaxis in some patients.

IgA on mucosal surfaces is produced locally and not derived from circulating IgA. IgA is one of the gamma globulins on the basis of its electrophoretic mobility. IgA is composed of two heavy chains and two light chains. It may be monomeric (i.e. a single molecule), dimeric or polymeric (composed of two or more monomers). IgA monomers are joined together as dimers at the constant regions of their heavy chains by a J chain. IgA is secreted as one of two subclasses, IgA1 and IgA2. IgA1 predominates in the circulating blood wherein most of it occurs as a monomer. Most IgA on mucosal surfaces, such as the surfaces of the trachea, bronchi, and bronchioles in the lungs, occurs as dimers or polymers joined by J chains. IgA dimers and polymers have an increased ability to bind to and agglutinate target molecules (antigens). Agglutinated antigens are more readily phagocytosed and thereby eliminated. In addition, IgA dimers and polymers, because of the presence of their J chains, have the ability to bind to secretory component. Recombinant expression of dimeric IgA with the incorporation of J chain of human origin has been accomplished (Johansen, et al., Eur J Immunol 1999; 29:1701-1708). Monomeric IgA interferes with influenza virus replication (Taylor, et al., J Exp Med 1985; 161:198-209) and polymeric IgA interferes with influenza binding to and entry into target cells (Taylor, et al. J Exp Med 1985; 161:198-209; Outlaw and Dimmack, J Gen Viral 1990; 71:69-76). Monomeric IgA interferes with influenza virus replication (Taylor, et al., J Exp Med 1985; 161:198-209). Human serum IgA with specificity against a given Coronaviridae has been recovered (positive control for a commercial given Coronaviridae IgA ELISA kit, (GSD SARS-CoV-2 ELISA IgA kit product number GSD01-1029, Eurofins Genomics LLC, Louisville, Ky. 40299).

Exogenous IgA has been topically applied to the nose in both animals and humans for the purpose of preventing and treating disease. In mice, nasal application of exogenous IgA has been demonstrated to be efficacious in protecting animals from influenza (Tamura, et al., Vaccine 1990; 8:479-485, Tamura, et al., Eur J Immunol 1991; 21:1337-1344), Sendai virus (Mazanec, et al., J Virol 1987; 61:2624-2626, Mazanec, etal., Virus Res 1992; 23:1-12) and respiratory syncytial virus (Weltzin, et al., Antimicrob Agents Chemother 1994; 38:2785-2791) challenge. Intranasal monoclonal IgA also protects rhesus monkeys against respiratory syncytial virus infection (Weltzin, et al., J Infect Dis 1996; 174:256-261). In humans, nasal administration of approximately 70% IgM 30% IgG resulted in decreased frequency of upper respiratory tract infections in elite skiers (Hemmingsson and Hammarstrom, Scand J Infect Dis 1993; 25:73-75), and in children (Giraudi, et al., Int J Pediatr Otorhinolarynol 1997; 39:103-110, Heikkinen, et al., Pediatr Infect Dis J 1998; 17:367-372) but not in elite canoeists (Lindberg and Berglund, Int J Sports Med 1996; 17:2335-238). Aerosol administration of human y globulin (Fruchtman, et al., Clin Med 1972 (September); 79:17-20), pooled human IgG (Rimensberger and Schaad, Pediatr Infect Dis J 1994; 13:328-330) and murine recombinant humanized IgG (Fahy, et al., Am J Respir Crit Care Med 1999; 160:1023-1027) demonstrated that there are no adverse effects from the aerosol inhalation of human y globulin or human or humanized IgG.

Individuals suffering from hypogammaglobulinemia or with bronchial infections from other causes have been treated by administration of antibiotics. However, antibiotic treatment is not completely effective in preventing infection in patients with immunoglobulin deficiency. Another method of treating such patients has been intravenous infusion of immunoglobulin G (IgG). The IgG is administered by intravenous infusion which may not reach the mucosal surface of the bronchial tree. In addition, intravenous infusion of immunoglobulin is usually administered by trained medical personnel and can be associated with systemic reactions.

Thus, there is a need for a method which can be used to deliver IgA to the bronchial, nasal and ocular mucosal surfaces, the IgA being anti-Coronaviridae immune IgA antibodies. It would be advantageous if such prophylaxis could be administered by the patient without the need for trained medical personnel. It would further be desirable to make use of unwanted by-products resulting from the preparation of purified immunoglobulin G from pooled human plasma. The present invention provides these advantages and others as will be apparent to one with skill in the art from the disclosure that follows.

### SUMMARY OF THE INVENTION

A method of inhibiting or treating infection by Coronaviridae virus of a susceptible host is provided that includes the administration of immune anti-Coronaviridae secretory IgA, having a recombinant secretory component. The administration of the immune anti-Coronaviridae secretory IgA is to tissues of the susceptible host. Exemplary target tissues include respiratory mucosa of the lung, bronchial tree, throat, or nose; or eyes. The administration being prior to, or after the susceptible host is exposed to the Coronaviridae virus. A composition for such administration is also provided.

### DETAILED DESCRIPTION OF THE PREFERRED EMBODIMENTS

The following description of the preferred embodiment(s) is merely exemplary in nature and is in no way intended to limit the invention, its application, or uses.

The present invention has utility in exposing superficial nasal, ocular and pharyngeal tissues to anti-Coronaviridae immune IgA antibodies prior to anticipated exposure to a Coronaviridae pathogen such as SARS-CoV or SARS-CoV-2 virus. Individuals such as first responders and medical personnel are able to anticipate exposure and apply protective anti Coronaviridae pathogen virus secretory IgA antibodies to mitigate the risk of infection.

The present invention provides a composition and method for the medical prevention in humans of Coronaviridae viral infections that involves topical mucosal administration by inhalation, nasal and throat spray, or ocular drops of an anti-Coronaviridae immune IgA antibodies. The composition may be administered intranasally as a powdered or liquid spray, nose drops, a gel or ointment, through a tube or catheter, by syringe, packtail, pledget, or by submucosal infusion. In one embodiment, the IgA is prepared as a by-product from pooled human plasma. In another embodiment, the IgA composition contains a monoclonal antigen-specific IgA. In still other inventive embodiments, the IgA component is further combined with recombinant secretory component to produce a more physiologically effective composition (secretory IgA).

While the present invention is detailed hereafter with respect to the currently pandemic SARS-Cov-2, it is appreciated that the present invention is readily used to confer resistance to infection by any Coronaviridae that infects its host via binding to nasal, ocular, or respiratory tissues of a host. It is appreciated that the host for the target coronavirus can be human or animal.

It is to be understood that in instances where a range of values are provided herein, that the range is intended to encompass not only the end point values of the range, but also intermediate values of the range as explicitly being included within the range and varying by the last significant figure of the range. By way of example, a recited range of from 1 to 4 is intended to include 1-2, 1-3, 2-4, 3-4, and 1-4.

In one embodiment, the invention provides a method for medical prophylaxis or treatment for SARS-CoV-2 in humans includes administering by inhalation an aerosol composition by nasal spray or by topical application to the ocular tissue. The administered composition contains an IgA component which can be derived from a number of sources. The composition contains an IgA component, anti-SARS-CoV-2 IgA antibodies, which can be derived from a number of sources including the by-product obtained from pooled human plasma following recovery of IgG and other plasma proteins to obtain. The IgA by-product is further purified as performed by those of skill in the art of protein purification, as for example detailed in U.S. Pat. No. 10,385,117B1.

In some inventive embodiments, the compositions contain, in addition to the dimeric and polymeric IgA component, a recombinant secretory component. Human secretory component can be produced by recombinant techniques as described in Croftet, et al, Biochem. J 1999; 341:299-306. In some inventive embodiments, the IgA is covalently bonded to the recombinant secretory component. In some inventive embodiments, the bonding is accomplished by forming disulfide bonds under mildly oxidizing conditions. Purified IgA containing secretory component can be stabilized for example by the addition of human serum albumin to a final concentration of 1 to 5%. The presence of the human secretory component in the compositions of the invention leads to inhaled doses of immunoglobulin A which are more physiologically effective than compositions without secretory components.

In another inventive embodiment, an IgA containing component is isolated as a by-product from anti-SARS-CoV-2 hyperimmune or immune pooled human plasma. Anti-SARS-CoV-2 hyperimmune or immune pooled human plasma is obtained from donors who have been immunized against SARS-CoV-2 or have recovered from infection with SARS-CoV-2, respectively. In another embodiment, the IgA component can be prepared by hybridoma techniques to provide antigen-specific IgA. Hybridoma techniques are described originally in Kohler and Milstein, Nature 1975; 256:495-497 with more recent advances summarized in Berzofsky et al., Fundamental Immunology, Third Edition, 1993, pp 455-462. Hybridoma production involves the fusion of an immortalized immunoglobulin-producing myeloma cell with an antibody producing cell from an immunized individual. The product is an immortalized cell culture which produces the specific antibody against the antigen that the donor individual was immune to. For example, a mouse monoclonal IgA antibody has been prepared against respiratory syncytial virus F glycoprotein as described in Weltzin, et al., J Infect Dis 1996; 174:256-261 and Weltzin, et al., Antimicrob Agents Chemother 1994; 38:2785-2791. In another inventive embodiment, the IgA is dimeric monoclonal IgA expressed already containing J chain. Monoclonal antibodies specific for the SARS-CoV-2 spike (S) protein by which the virus attaches to its target cells has been commercially produced (e.g. SARS-CoV-2 Si receptor binding domain antibodies for neutralization assays, antibodies-online, GmbH, Schloss-Rahe-Str. 15, 52072 Aachen, Germany and SARS-CoV-2 Spike Protein (S1), BioVision, Inc. 155 South Milpitas Blvd Milpitas, Calif. 95035). In another inventive embodiment, recombinant human secretory component is added to the polyclonal or monoclonal dimeric and polymeric J chain containing polyclonal IgA so that secretory IgA is synthesized. The secretory IgA is then used as a prophylaxis and/or treatment for SARS-CoV-2 infection by administration to the lungs by aerosol, to the nose and throat by spray and to the eyes by topical application of ophthalmic drops.

The compositions of the invention for pulmonary delivery of aerosol compositions generally contain in addition to the IgA component and optional J chains and secretory component known pharmaceutical excipients and buffering agents. Non-limiting examples of such excipients include proteins as for example, human serum albumin and recombinant human albumin. Other pharmaceutical excipients include carbohydrates, sugars, and alditols. Non-limiting examples of suitable carbohydrates include sucrose, lactose, raffinose, and trehalose. Suitable alditols include mannitol, and pyranosyl sorbitol. Polymeric excipients include polyvinylpyrolidone, Ficolls, soluble hydroxyethyl starch, and the like of suitable molecular weight. Non-limiting examples of suitable buffering agents include salts prepared from organic acids such as citric acid, glycine, tartaric acid, lactic acid, and the like. Other useful excipients include surfactants and chelating agents. The compositions of the invention are readily aerosolized and rapidly deposited in the lungs of a host. Doses are formulated from the compositions of the invention by combining the IgA component with or without human J chain and secretory component, and pharmaceutical excipients so as to contain an effective dose of the active ingredient. A typical dose would include 0.00001 to 5 milligrams of active material. The dose amount may be adjusted up or down as required to meet the prophylaxis needs of an individual, or to provide for ease and convenience in administering the dose. While dosing is typically once per day (qd), it is appreciated that other dosing regimes are readily applied for a given subject including twice (bid), three times (tid), four times (qid), or even more times per day.

The compositions of the invention can be administered by nasal spray, eye drops, by nebulization or by metered dose inhalers. Nebulizers and metered dose inhalers are well known in the art and are described for example, in Wolff and Niven, J Aerosol Med 1994; 7:89-106.

Pharmaceutically acceptable carriers, excipients, or diluents illustratively include saline, buffered saline, dextrose, water, glycerol, ethanol, sterile isotonic aqueous buffer, and combinations thereof.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

Materials for use in the preparation of microspheres and/or microcapsules include biodegradable/bioerodible polymers such as PLGA, polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, or lipoproteins.

Diseases and conditions for which aerosol pulmonary administration of the compositions of the invention is to be used therapeutically or prophylactically against a Coronaviridae infection.

As used here, the term therapeutic treatment means that the patient being administered a dose of a composition of the invention has been diagnosed as having the condition to be treated. Prophylaxis means that the patient is being treated to prevent infection. Such treatment is often indicated where a person is at risk of exposure.

The method of the present invention may be used with any animal, such as a mammal or a bird (avian). Exemplary mammals include, but are not limited to rats, mice, cats, dogs, horses, cows, sheep, pigs, non-human primates, and humans.

The compositions of the invention are readily formulated for delivery to a subject by topical application. Appropriate target areas for topical application depend on the goal of the preventative or therapeutic treatment and are readily determined by one skilled in the art and commonly include sites where local action is desirable. Generally, such target areas illustratively include the mouth, gastrointestinal tract, nose, sinuses, and eye and in particular, the mucosal surfaces of those areas. A target area as described above is further composed of target sub-areas which are treated separately from the whole target area. Formulations of IgA for topical application include concentrations ranging from 1 mg/ml to 600 mg/ml. In an embodiment of the present invention, a single topically applied dose of IgA ranges from 0.1 mg per cm² of target area to 600 mg per cm² of target area. In other inventive embodiments, the topically applied dose of IgA ranges from 1 mg per cm² of target area to 60 mg per cm² of target area. In still other inventive embodiments, the topically applied dose of IgA ranges from 2 mg per cm² of target area to 25 mg per cm² of target area. The single topical dose amount may be adjusted up or down, as required to meet the prophylaxis or treatment needs of an individual, or to provide for ease and convenience in administering the dose. The number of doses to be administered per day depends on the condition to be treated as well as the target area to which the composition is topically administered. In general, the number of doses per day ranges from 1 to 12, and in other inventive embodiments range from 1 to 4. The exact amount of the antibody as an agent required, will vary from subject to subject, depending on the age, weight and general condition of the subject, the severity of the disease that is being treated, the particular compounds used, the mode of administration, and the like. An appropriate amount may be determined by one of ordinary skill in the art using only routine experimentation given the teachings herein.

The compositions will include an effective amount of the selected agent in combination with a pharmaceutically acceptable carrier and, in addition, may include other medicinal agents, pharmaceutical agents, carriers, or diluents. By “pharmaceutically acceptable” is meant a material that is not biologically or otherwise undesirable, which can be administered to an individual along with the selected substrate without causing significant undesirable biological effects or interacting in a deleterious manner with any of the other components of the pharmaceutical composition in which it is contained.

### EXAMPLE 1

Polyclonal IgA is obtained from pooled anti-Coronaviridae virus human plasma following Cohn cold ethanol fractionation to produce Cohn fraction III precipitate or from pooled anti-Coronaviridae virus convalescent human serum or immunized healthy donor serum or plasma. IgA is further purified by jackbean lectin (jacalin) affinity chromatography or by ion exchange chromatography. Alternatively, monoclonal dimeric IgA expressed containing J chain is obtained from a hybridoma which produces dimeric IgA. In both cases the IgA is then coupled to recombinant secretory component again by disulfide bonding in mildly oxidizing conditions, preferably at a molar ratio of secretory component to IgA-J chain dimers and polymers of 1:1. The secretory IgA containing recombinant secretory component is again purified. Purified secretory IgA is stabilized by the addition of human serum albumin to a final concentration of 5%. The final solution, adjusted to a therapeutic dose of 5 mg IgA, is then placed in a nebulizer for self-administration. The resulting solution given at an equivalent of one dose per day is effective in inhibiting infection of human mucosa cells in vitro by SARS-CoV-2 virus.

### EXAMPLE 2

Polyclonal IgA is obtained from pooled anti-Coronaviridae virus human plasma following Cohn cold ethanol fractionation to produce fraction III precipitate. IgA is further purified by heparin-Sepharose adsorption, dextran sulfate and ammonium sulfate precipitation, hydroxyapatite chromatography, batch adsorption and by an anion-exchange matrix and gel permeation. Alternatively, monoclonal IgA is obtained from an IgA-producing hybridoma. The IgA is then coupled to recombinant secretory component again by disulfide bonding in mildly oxidizing conditions, preferably at a molar ratio of secretory component to IgA-J chain conjugates of 1:1. The synthesized secretory IgA containing solution is again purified. Purified secretory IgA is stabilized by the addition of human serum albumin to a final concentration of 5%. The final solution, adjusted to a therapeutic concentration of 20 mg/ml IgA in electrolyte solution containing 129 mEq/l Na⁺, 17 mEq/l K⁺, 0.32 mEq/l Ca⁺⁺, 0.35 mEq/l Mg⁺⁺, 0.11mEq/l Zn⁺⁺, 141 mEq/l Cl⁻ and 12 mEq/l bicarbonate, pH=7.7, is then self-administered with an eyedropper. The resulting solution given at one dose per day is effective in inhibiting infection of human mucosa cells by SARS-CoV-2 infection.

Patent applications and publications mentioned in the specification are indicative of the levels of those skilled in the art to which the invention pertains. These applications and publications are incorporated herein by reference to the same extent as if each individual application or publication was specifically and individually incorporated herein by reference.

The description of the invention is merely exemplary in nature and, thus, variations that do not depart from the gist of the invention are intended to be within the scope of the invention. Such variations are not to be regarded as a departure from the spirit and scope of the invention.

We claim:

1. A method of inhibiting or treating infection by Coronaviridae virus of a susceptible host comprising:
administering immune anti-Coronaviridae secretory IgA having a recombinant secretory component to the susceptible host; and
prior to, or after is host being exposed to the Coronaviridae virus.

2. The method of claim 1 wherein said immune anti-Coronaviridae secretory IgA is monoclonal.

3. The method of claim 1 wherein said immune anti-Coronaviridae secretory IgA is polyclonal.

4. The method of claim 3 wherein said immune anti-Coronaviridae secretory IgA is a by-product of the recovery of other plasma proteins from pooled plasma derived from more than one human individual, wherein the by-product is prepared by:
providing immune anti-Coronaviridae virus pooled human plasma;
fractionating the pooled human plasma to produce an IgA rich fraction;
adsorbing said IgA rich fraction onto an adsorptive medium to form a bound portion of said IgA;
recovering the bound portion of said IgA;
subjecting the recovered bound portion of said IgA to antiviral treatment; and
sterilizing said immune anti-Coronaviridae secretory IgA.

5. The method according to claim 4, wherein said pooled human plasma is derived from specifically Coronaviridae virus immunized donors.

6. The method according to claim 4, wherein the pooled human plasma is derived from donors specifically recovered from Coronaviridae virus infection.

7. The method according to claim 1 wherein the Coronaviridae virus is SAR-CoV-2 virus.

8. The method of according to claim 1 wherein said administering is prior to said exposing.

9. The method of according to claim 1 wherein said administering is after said exposing.

10. The method of according to claim 1 wherein said administering is by aerosol.

11. The method of according to claim 1 wherein said administering is by ophthalmic solution.

12. A composition comprising:
immune anti-Coronaviridae secretory IgA; and
a carrier for nasal, ocular, or oral delivery.

13. The composition of claim 12 wherein said immune anti-Coronaviridae secretory IgA is from pooled human plasma.

14. The composition of claim 12 wherein said immune anti-Coronaviridae secretory IgA prepared from recombinant human secretory component bound to the IgA natural dimer or higher polymer.

15. The composition of claim 12 wherein said immune anti-Coronaviridae secretory IgA prepared from recombinant human secretory component bound to recombinant monoclonal IgA dimer.

16. The composition of claim 12 wherein said immune anti-Coronaviridae secretory IgA is from pooled human convalescent serum.

17. The composition of claim 12 wherein said immune anti-Coronaviridae secretory IgA is from pooled immunized human plasma.

18. The composition of claim 12 wherein said immune anti-Coronaviridae secretory IgA is present from 1 to 50 mg/ml in said carrier.
